(19) United States
(12) Patent Application Publication
(10) Pub. No.: US 2011/0028321 A1
(43) Pub. Date: Feb. 3, 2011

(54) METHOD AND BACTERIUM FOR PROMOTING THE GROWTH OF RACOMITRIUM CANESCENS AND SEED PLANTS

(75) Inventors: Akio Tani, Okayama (JP); Motomu Akita, Wakayama (JP)

Correspondence Address: MILLEN, WHITE, ZELANO & BRANIGAN, P.C., 2200 CLARENDON BLVD., SUITE 1400, ARLINGTON, VA, 22201, US

(73) Assignee: National University Corporation Okayama University

(21) Appl. No.: 12/864,080

(22) PCT Filed: Jan. 23, 2009
(86) PCT No.: PCT/JP2009/051034

§ 371 Date: Oct. 18, 2010

(30) Foreign Application Priority Data
Jan. 23, 2008 (JP) 2008-012159
May 8, 2008 (JP) 2008-122462

(51) Int. Cl. A01N 63/00; C12N 1/20; C12N 5/04; A01P 21/00

(52) U.S. Cl. 504/117; 435/252.1; 435/410; 435/420

(57) ABSTRACT

Disclosed are bacteria and methods for promoting growth of Racomitrium canescens, tobacco, barley and soybean. The bacteria are selected from the methanol-utilizing bacteria deposited under Accession numbers FERM BP-11078, FERM BP-11079, FERM BP-11080 and FERM BP-11071, respectively which bacteria belong to the genus Methylobacterium. The methods are a method for promoting growth of protonemata of Racomitrium canescens which method is characterized by culturing the protonemata in the presence of the bacteria and a method for promoting growth of a seed plant selected from the group consisting of tobacco, barley and soybean which method is characterized by culturing seeds of the seed plant while contacting the seeds with the bacterium deposited under Accession number FERM BP-11078 which is one of the above-mentioned bacteria.

### TECHNICAL FIELD

The present invention relates to growth promotion of Racomitrium canescens and certain seed plants, more specifically, to bacteria having a property to promote growth of Racomitrium canescens and the seed plants, a composition comprising Racomitrium canescens and some of the bacteria, a method for promoting growth of Racomitrium canescens by letting Racomitrium canescens and the bacteria live symbiotically, and to a method for promoting growth of the seed plants by exposing their seeds to the bacterium.

### BACKGROUND ART

Though the bryophytes [Bryophyta] fix CO₂ and release oxygen through the process of carbon dioxide assimilation in the same manner as other plants (patent document 1), they, in the long run, have a higher CO₂ fixation ability than deciduous plants because while deciduous plants' fallen leaves are easily decomposed by microorganisms to release CO₂, the bryophytes, even after they die, are very slow to decay and therefore also very slow to release CO₂. So far, cultivation of bryophytes has been done under a condition where they are nearly left in the nature. However, this approach has a problem that their growth rate greatly depends on weather, and, further, a difficulty that their growth is so slow that it requires more than one year for planted bryophytes to form a community in the place where they were planted. Among bryophytes, Racomitrium canescens draws particular attention as greening material from such viewpoints that it is strong against dryness, grows even in a sunny place, requires no soil as a base, does not need maintenance, and the like (patent documents 2, 3, 4 and 5). However, it is the bottleneck against its wide use for greening that the growth of bryophytes is very slow. Consequently, there is a potential need for technology to promote growth of Racomitrium canescens.

Turning to other plants than the bryophytes, promoting growth of agriculture plants, for example, i.e., plants produced as food or animal feed, also leads to shorter time period until harvesting as well as to increased yield. Thus, such growth-promoting technology would be useful, and there is a potential need for the technology. It is known that Methylobacterium extorquens has a growth-promoting effect on Nicotiana tabacum, which is a species of tobacco (non-patent document 1), and that M. extorquens has a growth-promoting effect on barley (patent documents 6), and that M. extorquens also has a growth-promoting effect on soybean (patent documents 6-11). However, the promoting effects are not sufficient, and thus it is desirable that other bacteria having promoting effect become available.

[patent document 1] Japanese Patent Application Publication No. 2006-254900
[patent document 2] Japanese Patent Application Publication No. 2006-006292
[patent document 3] Japanese Patent Application Publication No. 2004-236518
[patent document 4] Japanese Patent Application Publication No. 2002-360060
[patent document 5] Japanese Patent Application Publication No. H07-227142
[patent document 6] U.S. Patent Application Publication No. 20030211082
[patent document 7] U.S. Pat. No. 5,512,069
[patent document 8] U.S. Pat. No. 5,961,687
[patent document 9] U.S. Pat. No. 6,174,837
[patent document 10] U.S. Patent Application Publication No. 20010001095
[patent document 11] U.S. Patent Application Publication No. 20060228797
[non-patent document 1] Journal of Experimental Botany, 57 (15), p. 4025-4032 (2006)

### DISCLOSURE OF THE INVENTION

### Problem to be Solved by the Invention

Against the above background, the present invention is intended to provide a means to promote growth of Racomitrium canescens and seed plants such as tobacco, barley and the like.

### Means to Solve the Problem

The present inventors assumed the presence of a symbiotic relation between Racomitrium canescens and other microorganisms, and have conducted a study seeking for such microorganisms. After having attempted to isolate bacteria from Racomitrium canescens collected, they have found the presence of methanol-utilizing bacteria. Symbiosis of Racomitrium canescens and methanol-utilizing bacteria has not been known so far. The inventors, assuming that the methanol-utilizing bacteria occur as dominant species for Racomitrium canescens, investigated what function they had on Racomitrium canescens. As a result, the inventors have found that there are some strains among the bacteria belonging to Methylobacterium aquaticum or Methylobacterium extorquens which act to promote growth of protonemata of Racomitrium canescens, and have isolated and identified those strains. It has not been known before that these two kinds of bacteria include strains which promote growth of protonemata of Racomitrium canescens. Further, the present inventors have found that in some seed plants, increase of germination rate or growth promotion of stem and root are brought about by contacting their seeds, before germination, with those bacterial strains for a period of time. Further, the inventors have found that some of those bacteria promote growth of seed plants. Based on these findings, the inventors have conducted a further study and completed the present invention. Namely, the invention provides the following.

1. A bacterium selected from methanol-utilizing bacteria of the genus Methylobacterium which bacteria are deposited under designated accession numbers FERM BP-11078, FERM BP-11079, FERM BP-11080 and FERM BP-11071, respectively.

2. A composition comprising bacterium of 1 above and protonemata of Racomitrium canescens.

3. A composition according to 2 above, wherein the bacterium and the protonemata are contained in a culture medium.

4. A method for growth promotion of protonemata of Racomitrium canescens, which method comprises a step of providing at least one of methanol-utilizing bacteria of the genus Methylobacterium which bacteria are deposited under designated accession numbers FERM BP-11078, FERM BP-11079, FERM BP-11080, and FERM BP-11071, respectively, and a step of culturing protonemata of Racomitrium canescens together with the provided bacterium or bacteria.

5. A method for growth promotion of a seed plant selected from tobacco, barley and soybean, which method comprises a step of providing Methylobacterium aquaticum of methanol-utilizing bacterium deposited under designated accession No. FERM BP-11078, and a step of contacting the seeds of the selected seed plant with the bacterium, and then culturing the seeds.

6. A method according to 5 above, wherein the contact of the seeds with the bacterium is conducted by exposing the seeds to the culture medium of the bacterium.

### EFFECT OF THE INVENTION

According to the present invention, growth of protonemata of Racomitrium canescens can be promoted. Consequently, according to the invention, it enables to overcome the difficulty of Racomitrium canescens that it is slow to grow, and thus to grow the protonemata quickly. Also, the invention, by promoting germination of their seeds or growth of their stems and roots, of tobacco and barley, enables to promote growth of these seed plants. Consequently, by using the invention for agriculture, it is possible to shorten a time period until harvest or to increase the yield.

### BRIEF DESCRIPTION OF DRAWINGS

FIG. 1 is a photograph substituted for a drawing, showing the results of DGGE.

FIG. 2 is photographs substituted for a drawing, showing growth comparison results of protonemata by types of and with or without inoculation of bacteria.

FIG. 3 is a graph showing growth comparison results of protonemata by types of and with or without inoculation of bacteria.

FIG. 4 is a photograph substituted for a drawing, showing growth inhibition of molds by MA-22A.

FIG. 5 is photographs substituted for a drawing, showing how tobacco has grown observed from above of plant boxes.

FIG. 6 is graphs showing how tobacco has grown from seed.

FIG. 7 is graphs showing how barley has grown from seed.

FIG. 8 is a graph showing how soybean has grown from seed.

FIG. 9 is a graph showing how soybean has grown from seed.

### BEST MODE FOR CARRYING OUT THE INVENTION

Among the methanol-utilizing bacteria of the genus Methylobacterium of the present invention, those named MA-22A, MC-21B and MC-21C were deposited domestically on Nov. 28, 2007 to International Patent Organism Depositary (IPOD), National Institute of Advanced Industrial Science and Technology, Independent Administrative Agency, at Tsukuba Center Chuo No. 6, 1-1-1, Higashi, Tsukuba-shi, Ibaraki, Japan, and accession numbers of FERM P-21449, FERM P-21450 and FERM P-21451 were designated thereto, respectively, and then, those deposits were changed to international deposits based on the Budapest Treaty on Dec. 16, 2008 at IPOD, and accession numbers of FERM BP-11078, FERM BP-11079 and FERM BP-11080 were designated thereto, respectively. Also, bacterium named MC-11A was deposited internationally to IPOD on Dec. 10, 2008, and accession number of FERM BP-11071 was designated thereto.

Properties of MA-22A, MC-21B, MC-21C and MC-11A are shown in the following Table 1a and Table 1b.

TABLE 1a
Deposited Bacterial Strains

| Name of Strain | MA-22A | MC-21B | MC-21C |
|---|---|---|---|
| Name of | Methylobacterium | Methylobacterium | Methylobacterium |
| Bacterium | aquaticum | extorquens | extorquens |
| Designated | FERM BP-11078 | FERM BP-11079 | FERM BP-11080 |
| Accession | | | |
| Number | | | |
| Oxygen | aerobic | aerobic | aerobic |
| Requirement | | | |
| Light | unnecessary | unnecessary | unnecessary |
| Requirement | | | |
| Colony | Color: pink | Color: pink | Color: pink |
| Morphology | Form: circular | Form: circular | Form: circular |
| | Bulging State: | Bulging State: | Bulging State: |
| | hemispheric | hemispheric | hemispheric |
| | Circumference: | Circumference: | Circumference: |
| | entire | entire | entire |
| | Surface: smooth | Surface: smooth | Surface: smooth |
| | Transparency: | Transparency: | Transparency: |
| | opaque | opaque | opaque |
| | Viscosity: butter | Viscosity: butter | Viscosity: butter |
| | like | like | like |
| | Change of Colony | Change of Colony | Change of Colony |
| | Morphology by | Morphology by | Morphology by |
| | Mutation: non | Mutation: non | Mutation: non |
| | Change of Colony | Change of Colony | Change of Colony |
| | Morphology by | Morphology by | Morphology by |
| | Culturing | Culturing | Culturing |
| | Condition/ | Condition/ | Condition/ |
| | Physiological | Physiological | Physiological |
| | Condition: non | Condition: non | Condition: non |
| Methanol- | + | + | + |
| utilizing | | | |
| Ability | | | |

TABLE 1b
Deposited Bacterial Strains

| Name of Strain | MC-11A |
|---|---|
| Name of | Methylobacterium |
| Bacterium | extorquens |
| Designated | FERM BP-11071 |
| Accession | |
| Number | |
| Oxygen | aerobic |
| Requirement | |
| Light | unnecessary |
| Requirement | |
| Colony | Color: pink |
| Morphology | Form: circular |
| | Bulging State: |
| | hemispheric |
| | Circumference: |
| | entire |
| | Surface: smooth |
| | Transparency: |
| | opaque |
| | Viscosity: butter |
| | like |
| | Change of Colony |
| | Morphology by |
| | Mutation: non |
| | Change of Colony |
| | Morphology by |
| | Culturing |
| | Condition/ |
| | Physiological |
| | Condition: non |
| Methanol- | + |
| utilizing | |
| Ability | |

Each bacterium above is that which was isolated from Racomitrium canescens in nature. In order to use these bacteria for growth promotion of protonemata of Racomitrium canescens, it is enough, for example, to prepare in a medium at least one of these bacteria before culturing protonemata of Racomitrium canescens, and then inoculate the protonemata with the bacterium or bacteria. In this case, the culture may be performed, for example, using the Culture Medium Y (including 0.8% agar) described in the part of the Examples in a light place (e.g., under a fluorescent lamp) at high humidity (e.g., 100%) and at 20° C., though the condition is not limited thereto, and culture may be performed under an appropriate condition. The amount of bacterium or bacteria with which the protonemata of Racomitrium canescens were inoculated may be determined as desired with no particular limitation. For example, growth promotion is observed by inoculating 1/30 of bacteria quantity in a colony (approximately 1 mm in diameter) per protonema. Since these bacteria easily proliferate when they are in the vicinity of protonema of Racomitrium canescens, the quantity to be inoculated may be less than this as far as subsequent growth of the bacteria is observed. Each of the above bacteria is pink-colored, and their growth can be clearly observed with the naked eye as a pink region surrounding a protonema. As far as growth of inoculated bacteria can be observed through culture, only the first inoculation is enough, though additional inoculation may be made as desired. A large quantity of protonemata to be let differentiate into gametophytes can be obtained in a short period of time by making protonemata quickly grow in this way.

In order to promote growth of spermatophytes (tobacco or barley), when germinating their seeds under normal germination conditions which are suitable for the seeds, it is enough to prepare, among the above bacteria, MA-22A (viable bacterial cells or then culture), contact them with the seeds, and to culture them in a usual manner. Usually, there is no need of latter additional inoculation to the culture medium because the inoculated bacteria proliferate in the culture process of the seeds. As far as letting the bacteria exist together with the seeds for a certain period of time (e.g., more than five days), increase of germination rate or other growth promotion can be achieved.

### EXAMPLES

This invention is described below in detail based on examples, though it is not intended that the invention be limited to the examples.

### Example 1

### Growth Promotion of Racomitrium canescens (1)

### Sterilization of Racomitrium canescens

Naturally grown moss of Racomitrium canescens (grown in Yamagata Prefecture) was obtained. The sporangia were washed with water of sufficient quantity (more than 1 mL per sporangium) twice, and treated with 70% ethanol for one minute. After having been further immersed in an aqueous solution containing 1% sodium perchlorate and 0.5% Tween20 for 20 minutes, the sporangia were washed with sterilized water five times. The sporangia were broken in sterilized water, and spores flowing out were collected with pipette. Sterile spores thus obtained were spread to an agar culture medium (a liquid of Hyponex™* diluted 1000 folds with water+0.8% agar), and cultured under a fluorescent lamp at 20° C. Growth of protonemata was observed with the naked eye in approximately two weeks after the start of culture. Note: Hyponex [mfd. by HYPONEX JAPAN CORP., LTD, containing 6% nitrogen in total (of which, 2.90% ammonium-nitrogen, 1.05% nitrate-nitrogen), 10.0% water soluble phosphate, 5.0% water soluble potassium, 0.05% water-soluble magnesia, 0.001% water-soluble manganese, 0.005% water-soluble boron]

The culture was performed also in the following Culture Medium Y (containing 0.8% agar), and growth of protonemata was equally observed with the naked eye. These sterile protonemata were used in experiments of growth promotion by microorganisms.

TABLE 2
Composition of Culture Medium Y

| Component | Quantity Contained (mM) |
|---|---|
| NH₄NO₃ | 1.25 |
| KCl | 2.68 |
| CaCl₂/2H₂O | 1.77 |
| MgSO₄/7H₂O | 0.81 |
| KH₂PO₄ | 1.47 |
| Na₂EDTA | 0.181 |
| FeSO₄/7H₂O | 0.09 |
| H₃BO₃ | 0.024 |
| MnSO₄/7H₂O | 0.099 |

[Search for Microorganisms from Racomitrium canescens]

The above naturally grown moss of Racomitrium canescens (before sterilization) was suspended in sterilized water, added to 1 L of liquid culture medium (Hyponex™ diluted 1000 folds with water), and then stirred at approximately 18° C. for seven weeks under a fluorescent lamp (30 cm distance, 16 hours/day). Each sample of culture medium was collected before stirring, after four and seven weeks of stirring. Each sample obtained was cultured using plates made of ⅓ LB agar medium (3.3 g/L polypeptone, 1.6 g/L yeast extract, 10 g/L sodium chloride, 15 g/L agar) and plates of 1000 fold-diluted Hyponex which was solidified with agar (1.5%). A number of various colonies were isolated, and each of them was subjected to identification of bacteria and to examination of their property. However, none of the bacteria was found to promote growth of Racomitrium canescens.

Then, the above naturally grown moss of Racomitrium canescens (before sterilization) was suspended in sterilized water, and cultured under each of the above culture conditions. The culture was sampled at random four and seven weeks after the start of the culture, and then subjected to DGGE (denaturing gradient gel electrophoresis) analysis. That is, 5 mL of each culture were centrifuged (10,000×g, ten minutes) into precipitate and supernatant. The precipitate was collected and washed with a 0.85% sodium chloride solution, and, then, suspended in one mL of BL buffer [40 mM Tris-HCl, 1% Tween20, 0.5% Nonidet P-40 (mfd. by Nacalai Tesque Inc.), 1 mM EDTA2Na, pH 8.0]. The suspension was frozen at −20° C., and subsequently melted at 60° C. 10 mL of proteinase K solution (10 mg/mL) was added thereto. The mixture was allowed to stand at 37° C. for 10 minutes, and PCR reaction was carried out in the following manner using the mixture as DNA templates.

TABLE 3
Composition of PCR Reaction Fluid

| Component | Quantity Contained (μL) |
|---|---|
| DNA Templates | 2.5 |
| 10× Ex taq Buffer | 5 |
| dNTP Mixture | 4 |
| Primer F(10 pmol/μL)* | 2.5 |
| Primer R(10 pmol/μL)* | 2.5 |
| Ex TaqDNA Polymerase | 0.25 |
| Water | 50 in total |

Note: Primer F:
(SEQ ID NO: 1)
CGCCCGCCGCGCGCGGCGGGCGGGGCGGGGGCACGGGGGGACTCCTACGG
GAGGCAGCAG
Note: Primer R:
(SEQ ID NO: 2)
ATTACCGCGGCTGCTG

### <Temperature Conditions>

(1) 95° C., two minutes
(2) 35 cycles of the following

95° C., one minute

55° C., 30 seconds

72° C., 30 seconds

(3) 72° C., five minutes

### <DGGE Condition>

According to the manual of the Dcode Universal Mutation Detection System (mfd. by Bio-Rad), electrophoresis was conducted as follows.

Gel: 9% acrylamide gel
Denaturing gradient: 40-65%

### Voltage: 60V

Time for electrophoresis: 17 hours

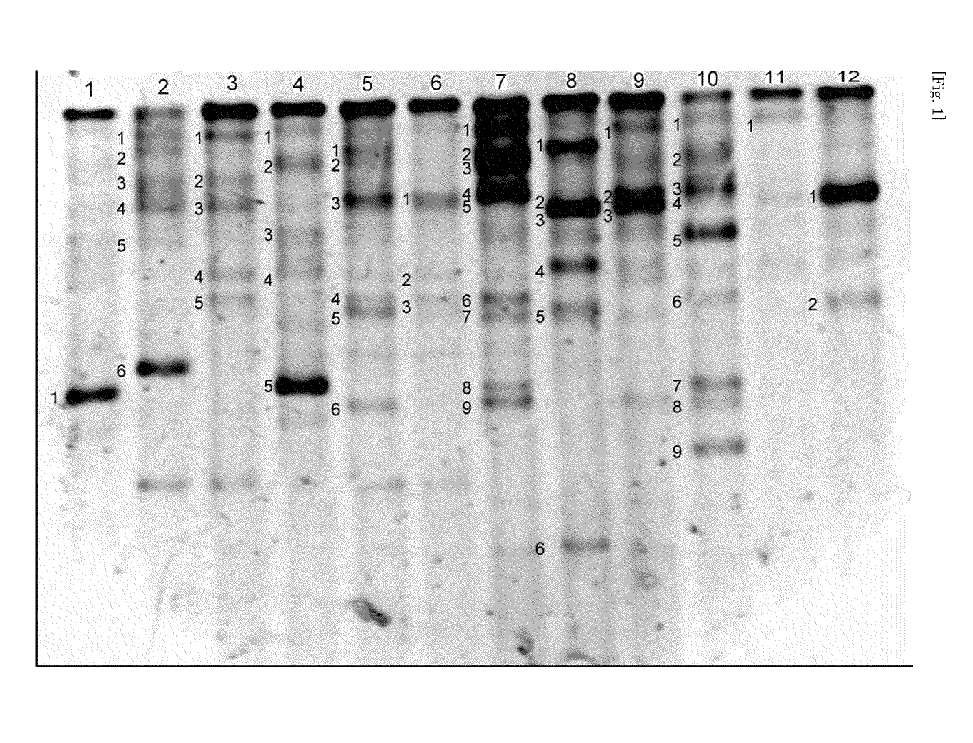

The result of electrophoresis is shown in FIG. 1, and the search result using database search based on the sequence of DNA fragments obtained is shown in Table 4, respectively.

TABLE 4
Attribution of Bands (Lane No. - Band No.)

```
  1-1 Arthrobacter oxydans
  2-1 Brevibacillus thermoruber
  2-4 Sphingomonas sp.
  2-5 Hirschia baltica
  2-6 Spirosoma linguale
  3-4 Methylobacterium sp.
  4-3 Micrococcus luteus
  4-5 Micrococcus indicus
  5-3 Methylocella palustris
  5-4 Micrococcus indicus
  5-5 Achromobacter sp.
  5-6 Xanthobacter aminooxidans (Low homology)
  7-1 Spirosoma-like sp.
  7-5 Acinetobacter sp.
  7-6 Achromobacter sp.
  7-7 Phaeospirillum molischianum
  7-8 Sanguibacter sp.
  7-9 Streptomyces thermovulgaris
  8-1 Cytophaga hutchinsonii
  8-2 Cohnella yongneupensis or Exiguobacterium indicum
  8-5 Aquaspirillum sinuosum (Low homology)
  8-6 Methylobacterium sp.
  9-1 Hirschia baltica
  9-2 Methylocella palustris
  9-3 Spirulina sp. (Low homology)
10-1 Sphingopyxis sp.
10-2 Bacillus sp.
10-3 Methylocapsa acidiphila
10-4 Methylocella palustris
10-5 Acinetobacter sp., and others
10-6 Dyadobacter hamtensis
10-7 Arthrobacter oxydans
10-9 Dyadobacter hamtensis
11-1 Spirosoma linguale
12-1 Spirulina sp. (Low homology)
12-2 Polaromonas sp.
```

As a result, it was found that a number of methanol-utilizing bacteria such as Methylobacterium, Methylocella, Methylocapsa and the like, which were not observed by the above culture, were included. As for these methanol-utilizing bacteria, there is no report so far, to the knowledge of inventors, that they have been detected in Racomitrium canescens. The reasons for that seem that methanol-utilizing bacteria hardly grow in a culture medium rich in nutrients, and their growth therefore is slower than other bacteria. The fact that methanol-utilizing bacteria have been detected in Racomitrium canescens by the DGGE method suggests that methanol-utilizing bacteria are present preferentially with Racomitrium canescens.

According to the above result, in order to collect methanol-utilizing bacteria present with Racomitrium canescens, the above each sample of Racomitrium canescens was suspended in sterilized water, and inoculated to a culture medium which contained methanol as the carbon source (Table 5, referred to herein as “methanol culture medium”), and isolation of bacterial colony was attempted.

TABLE 5
Composition of Culture Medium for Methanol-
utilizing Bacteria (in 1 ml)

| Component | Quantity Contained | |
|---|---|---|
| (NH₄)₂HPO₃ | 0.3 | mg |
| Potassium Chloride | 0.1 | mg |
| Yeast Extract | 0.05 | mg |
| 1% MgSO₄/7H₂O | 10 | μL |
| Vitamin Solution (Table 4) | 10 | μL |
| Metal Solution (Table 5) | 10 | μL |

| | |
|---|---|
| Methanol | 1%(after autoclaved) |

TABLE 6
Composition of Vitamin Solution

| Component | Quantity Contained (g/L) |
|---|---|
| Calcium Panthothenate | 0.4 |
| Inositol | 0.2 |
| Nicotinic Acid | 0.4 |
| p-aminobenzoic acid | 0.2 |
| Pyridoxine Hydrochloride | 0.4 |
| Thiamine Hydrochloride | 0.4 |
| Biotin | 0.2 |
| Vitamin B₁₂ | 0.2 |

TABLE 7
Composition of Metal Solution (in 1 L)

| Component | Quantity Contained (g/L) |
|---|---|
| CoCl₂/6H₂O | 1.9 |
| MnCl₂/6H₂O | 1.0 |
| ZnCl₂ | 0.7 |
| H₃BO₃ | 0.06 |
| Na₂MoO₄/2H₂O | 0.36 |
| NiCl₂/6H₂O | 0.24 |
| CuCl₂/2H₂O | 0.02 |

As a result of the above culture, total seven strains were isolated as methanol-utilizing bacteria. 16S rDNA of each strain was analyzed, and the results were compared with those of strains which are open to the public on databases. Genus and species of each strain and sequence of each 16S rDNA are shown. (Provided that, only several hundred bases of the first half were determined for 21A and 41A.)

(1) MC-11A: Methylobacterium extorquens, 16S rDNA sequence: SEQ ID NO:3

(2) MC-11B: Methylobacterium extorquens, 16S rDNA sequence: SEQ ID NO:4

(3) 21A: It is closely related to bacteria of genus Spirosoma, 16S rDNA: SEQ ID NO:5

(4) MC-21B: Methylobacterium extorquens, 16S rDNA sequence: SEQ ID NO:6

(5) MC-21C: Methylobacterium extorquens, 16S rDNA sequence: SEQ ID NO:7

(6) MA-22A: Methylobacterium aquaticum, 16S rDNA sequence: SEQ ID NO:8

(7) 41A: It is closely related to bacteria of genus Mesorhizobium, 16S rDNA: SEQ ID NO:9

[Study on Effect of the Isolated Bacteria on Growth of Racomitrium canescens]

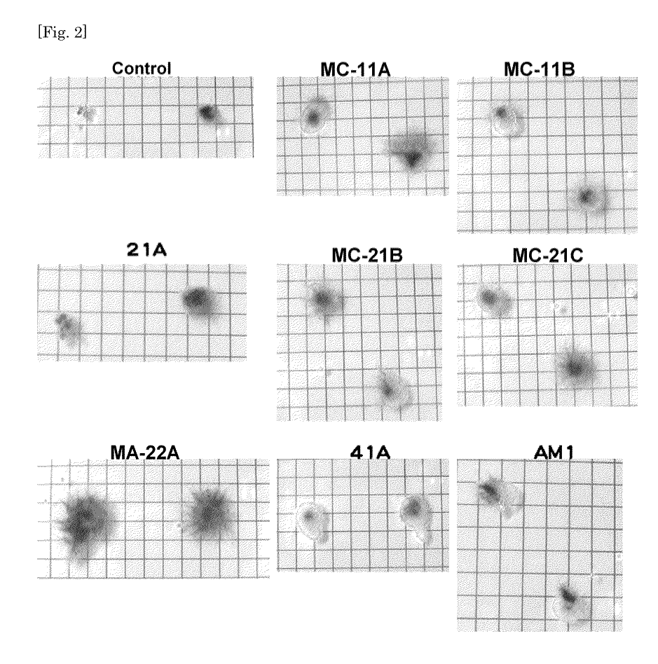

Protonemata which had been grown in Culture Medium Y (containing 0.8% agar) beforehand were picked up with tweezers one by one, and transferred onto two points of fresh Culture Medium Y (containing 0.8% agar). Colonies (approximately 1 mm in diameter) of the above bacteria which had been grown on the methanol agar medium were collected with a sterilized platinum loop, and suspended in 300 μL of sterilized water, and 10 μL each of the suspension were inoculated to the planted protonemata, and then the medium was cultured under a fluorescent lamp at 20° C. For purpose of comparison, the bacterium of Methylobacterium extorquens AM1 strain (ATCC14718) obtained from the culture collection was inoculated to protonemata in the same manner. Also, as a control, protonemata without inoculation of bacteria were cultured in the same manner. Results of 40 days of cultivation are shown in FIG. 2. As seen in the figure, evident promotion of growth of the Racomitrium canescens was observed which had been inoculated with MA-22A, MC-21B or MC-21C when compared to the control, and the promotion of growth was particularly remarkable in moss inoculated with MA-22A. In contrast, growth promotion was not seen in protonemata inoculated with AM1 strain which was one of already known strains of Methylobacterium extorquens. Further, degree of growth was digitized in terms of elliptical area surrounding each protonemata at time points of 3 weeks, 5 weeks and 9 weeks after the start of the study, for each protnemata inoculated with MC-11A, MC-11B, MC-21B, MC-21C and MA-22A and protonemata without inoculation of bacteria (control) (FIG. 3) (Error bars represent standard deviation. The same also applies in other graphs.) As a result, it was confirmed again that growth of protonemata inoculated with MA-22A is particularly outstanding, and it was also revealed that among other bacteria, MC-11A and MC-11B as well as MC-21B and MC-21C have growth-promoting effect.

### [Study on Characteristics of Isolated Bacteria]

The separated bacteria were studied on properties which were considered to be important in interactions with plants. That is, nitrogen fixation ability, siderophore (siderophore: a ferric chelating substance) secretion, ability of indoleacetic acid synthesis from tryptophan (Trp), dissolution ability of insoluble calcium phosphate, beta-glucanase secretion ability, presence of the HCN (which has growth inhibitory effect on moulds) secretion ability, and presence of growth inhibitory effect on moulds were examined concerning the separated bacteria, as follows.

### <Testing Method>

### (1) Nitrogen Fixation Ability:

Bacteria were streaked on an agar medium of the following composition which did not include nitrogen source, and growth was observed at 28° C. in the dark. Nitrogen fixation ability was determined to be positive if growth was observed.

TABLE 8
Composition of Agar Medium without
Nitrogen Source (in 1 L, pH 7.0)

| Component | Quantity Contained | |
|---|---|---|
| K₂HPO₄ | 1 | g |
| MgSO₄/7H₂O | 0.2 | g |
| CaCO₃ | 1 | g |
| NaCl | 0.2 | g |
| FeSO₄/7H₂O | 5 | mg |
| Agar | 15 | g |

| | |
|---|---|
| Glucose | 10 g (sterilized separately; and added |
| | after autoclaved) |

### (2) Siderophore Secretion:

According to Anal. Biochem, 160, 47-56, 1987, a test was conducted as follows. That is, at first, the following Solutions (A) to (C) were prepared.

(A) 60.5 mg of CAS (chrome azurol 5) was dissolved in 50 mL of water, and mixed with 10 mL of 1 mM FeCl₃ solution in 10 mM hydrochloric acid. To the solution thus prepared was added slowly a solution of 72.9 mg of HDTMA (hexadecyltrimethylammonium bromide) dissolved in 40 mL of water.

(B) 750 mL of Water, 100 mL of 10×MM9 salts (KH₂PO₄: 3 g/L, NH₄Cl: 10 g/L, NaCl: 5 g/L, MgSO₄: 2 mM, CaCl₂: 1 mM), 15 g of agar, and 30.24 g of PIPES [piperazine-1,4-bis(2-ethanesulfonic acid)] were mixed, and the pH was adjusted to 6.8 with sodium hydroxide aqueous solution.

(C) 30 mL of 10% casamino acid aqueous solution.

After the above Solutions (A) to (C) were separately sterilized by autoclaving, cooled to about 50° C., and mixed with each other. To this was added methanol at the final concentration of 1%, and the mixture was allowed to solidify in petri dishes. The plates thus obtained were blue in color. On the plates were streaked bacteria to be tested, and their growth was observed (at 28° C., in the dark). In this culture medium, siderophore-positive bacteria will form colonies, the areas around which will lose their blue color and turn pink or transparent. Siderophore-negative bacteria will not grow in this culture medium.

(3) Ability of Indoleacetic Acid Synthesis from Tryptophan:

According to Applied environmental microbiology, 1995, 61, 793-796, a test was conducted as follows. That is, bacteria to be tested were inoculated to King's B liquid culture medium (Difco proteose peptone No. 3: 20 g/L, K₂HPO₄: 1.15 g/L, MgSO₄: 1.5 g/L, methanol: 1%, tryptophan: 2.5 mM). The culture medium was cultured with shaking at 28° C. in the dark. After growth was observed, the culture was centrifuged, and the supernatant was obtained as a sample. To one mL of the sample, one mL of Solution R1 (FeCl₃ in 7.9M H₂SO₄: 12 g/L) was added. The mixture was left to stand at room temperature for 30 minutes, and then, absorbance at 530 nm was measured. A culture medium without inoculation of bacteria was used as a blank, and indoleacetic acid synthetic ability was determined as positive if color appears in comparison with the blank.

### (4) Dissolving Ability of Insoluble Calcium Phosphate:

According to FEMS Microbiol, Lett, 170, 265, 1999, a test was conducted as follows. That is, using NBRIP culture medium [National Botanical Research Institute's phosphate growth medium: glucose 10 g/L, Ca₃(PO₄)₂: 5 g/L, MgCl₂: 5 g/L, MgSO₄: 0.25 g/L, KCl: 0.2 g/L, (NH₄)₂SO₄: 0.1 g/L, pH 7, 1.5% agar], bacteria to be tested were streaked on the agar medium, and their growth was observed. The culture medium is cloudy (calcium phosphate), and growth and loss of the color of the medium will be observed if the bacteria have dissolving ability of calcium phosphate.

### (5) β-Glucanase Secretion Ability:

Azurin-dyed and cross-linked beta-glucan was added at the concentration of 0.2% to commercially available R2A agar medium, and the mixture was allowed to solidify after autoclaving. The culture medium turned blue, but the substrate was not dissolved. Bacteria to be tested were streaked on the medium, and judged as positive if dispersion of dissolved substrate was observed around colonies.

### (6) HCN Secretion Ability:

According to MPMI vol 16, 2003, 525-535, a test was conducted as follows. That is, bacteria to be tested were inoculated to Castric's culture medium (Can J. Microbiol. 21, 613-618, 1975, L-glutamic acid: 40 mM, glycine: 10 mM, L-methionine: 10 mM, MgSO₄: 2 mM, NaH₂PO₄: 5 mM, K₂HPO₄: 5 mM, FeCl₃: 20 μM, pH 7.0), cultured with shaking at 28° C. in the dark. Test tubes which could be capped were used. After growth of bacteria was observed, the culture was centrifuged, and the supernatant was diluted 10 folds with water, and 50 μL of this dilution was mixed with 250 μL of 0.1M NaOH. To this was added 0.5 mL of 0.1M o-dinitrobenzene solution in 2-methoxyethanol, and, after 15 seconds, 0.5 mL of 0.2M 4-nitrobenzaldehyde in 2-methoxyethanol was added, and then, 30 minutes later, absorbance at 578 nm was measured. Simultaneously, 1-16 μM KCN solutions were treated in the same manner, and were made standard.

### <Results>

The results of the tests are shown in the following Table.

TABLE 9

| | | | | Ability of | Dissolution | | |
|---|---|---|---|---|---|---|---|
| | | | | Indoleacetic | Ability of | | |
| | | Nitrogen | | Acid | Insoluble | β-glucanase | HCN |
| | | Fixation | Siderophore | Synthesis | Calcium | Secretion | Secretion |
| Strain | Attribution | Ability | Secretion | from Trp | Phosphate | Ability | Ability |
| MC-11A | Methylobacterium | + | + | + | + | − | + |
| (FERM | extorquens | | | | | | |
| BP-11071) | | | | | | | |
| MC-11B | Methylobacterium | + | + | + | + | − | + |
| | extorquens | | | | | | |
| MC-21B | Methylobacterium | + | + | + | + | − | + |
| (FEFM | extorquens | | | | | | |
| BP-11079) | | | | | | | |
| MC-21C | Methylobacterium | + | − | + | − | − | + |
| (FERM | extorquens | | | | | | |
| BP-11080) | | | | | | | |
| MA-22A | Methylobacterium | + | − | + | − | − | ++ |
| (FERM | aquaticum | | | | | | |
| BP-11078) | | | | | | | |
| 41A | Mesorhizobium | + | − | − | + | − | − |
| | sp. | | | | | | |
| 21A | Spirosoma sp. | + | − | − | − | + | − |
| AM1 | Methylobacterium | + | + | + | + | − | not tested |
| (ATCC | extorquens | | | | | | |
| 14718) | | | | | | | |

### (7) Growth Inhibition Effect on Moulds:

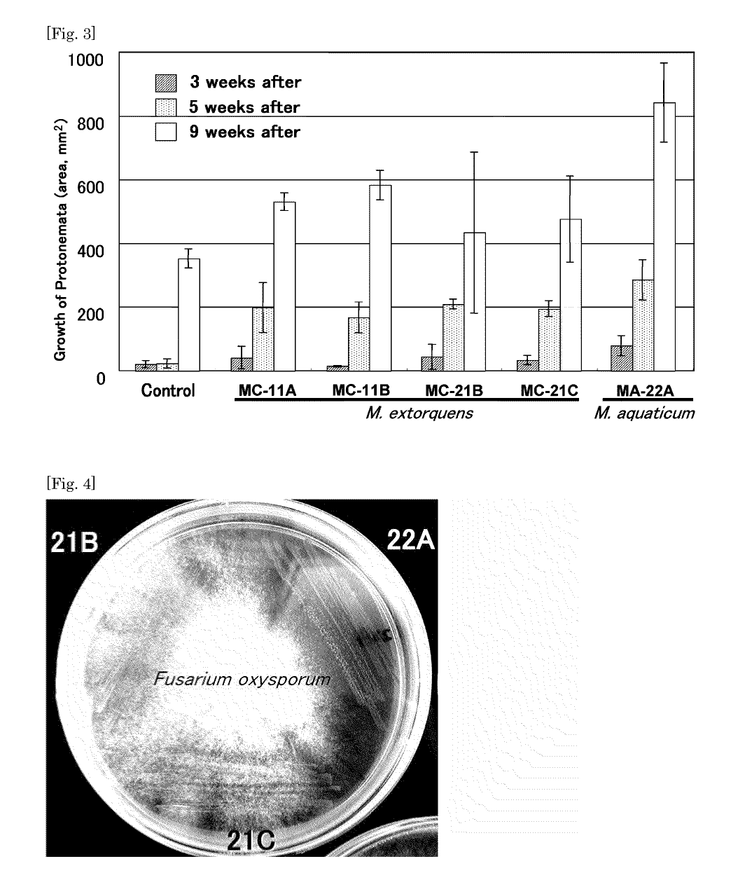

White filamentous mould which grew on the surface of Racomitrium canescens were separated, and identified as Fusarium oxysporum by sequencing 18S rRNA genes according to a conventional method. This mould was planted on the middle of a R2A agar (Beckton-Dickinson and Company) plate. Isolated MA-22A, MC-21B and MC-21C were streaked around the mold planted, respectively, and the plate was cultured at 28° C. for about 5 days. It was found that bacterium MA-22A strongly inhibited the mold hyphae from spreading over the agar. The state is shown in FIG. 4.

### Example 2

### Growth-Promotion of Tobacco

Seeds of tobacco (Nicotiana tabacum and Nicotiana benthamiana: provided from Leaf Tobacco Research Laboratory, Japan Tobacco Inc.) were treated with 70% ethanol for one minute, and then with 2% hypochlorous acid solution (containing approximately 1% Tween20) for 20 minutes. Seeds were washed 4-5 times with sterilized water. Four pieces of filter paper cut in 5.5 cm square were placed in plant boxes (made of plastic, 5.5 cm square, 9 cm of height), and sterilized by autoclaving. To this then was added 5 mL each of sterilized ½ Murashige-skoog culture medium (½ MS culture medium containing 0.8% agar). The Murashige-skoog liquid culture medium is of the composition shown in Tables 10-14, and ½ MS culture medium is provided by diluting it into ½.

Seeds were placed on the filter paper (30 seeds/plant box). Then, one of the cultures of MC-21, MC-21C and MA-22A strains which had been cultured (at 28° C., for 2-3 days) in 5 mL of methanol culture media (Table 5) (containing 1% methanol) was applied (500 μL/plant box) on those seeds. Groups of seeds on which only water or methanol, instead of the culture medium of bacteria, was applied were prepared as control. After the seeds were left to stand at 4° C. for 4 days in the dark in order to let the seeds absorb water, culture was performed at 25° C. for 15 days under light of a fluorescent lamp for 12 hours per day and in the dark for the rest of 12 hours. Length from seeds to cotyledons (stem), length of roots, and weight of seedlings were measured. Also, before the measurement, the state of their growth was photographed from above of the plant boxes.

TABLE 10
MS Liquid Culture Medium (pH 5.8, in 100 mL)

| Component | Quantity Contained (mL) |
|---|---|
| Solution 1 (MS macro) | 10 |
| Solution 2 (MS micro) | 0.1 |
| Solution 3 (EDTA-Fe) | 1 |
| Solution 4 (MS Vitamin) | 0.5 |

Sucrose is not contained

TABLE 11
Solution 1 (MS macro) 10-Fold Solution (in 100 mL)

| Component | Quantity Contained (mg) |
|---|---|
| Ammonium Nitrate | 1650 |
| Potassium Nitrate | 1900 |
| Calcium Chloride Dihydrate | 440 |
| Magnesium Sulfate Heptahydrate | 370 |
| Potassium Dihydrogenphosphate | 170 |
| Manganese Sulfate Tetrahydrate | 22.3 |
| Zinc Sulfate Tetrahydrate | 8.6 |
| Boric Acid | 6.2 |

TABLE 12
Solution 2 (MS micro) 1000-Fold Solution (in 100 mL)

| Component | Quantity Contained (mg) |
|---|---|
| Cobalt Chloride Hexahydrate | 2.5 |
| Copper Sulfate Pentahydrate | 2.5 |
| Sodium Molybdate Dihydrate | 25 |
| Potassium Iodide | 83 |

TABLE 13
Solution 3 (EDTA-Fe) 100-Fold Solution (in 100 mL)

| Component | Quantity Contained (mg) |
|---|---|
| Ferrous Sulfate Heptahydrate | 278 |
| Na₂-EDTA | 373 |

TABLE 14
Solution 4 (Vitamin) 200-Fold Solution (in 100 mL)

| Component | Quantity Contained (mg) |
|---|---|
| Nicotioic Acid | 10 |
| Pyridoxine Hydrochloride | 10 |
| Thiamine Hydrochloride | 2 |
| Inositol | 2000 |
| Glycine | 40 |

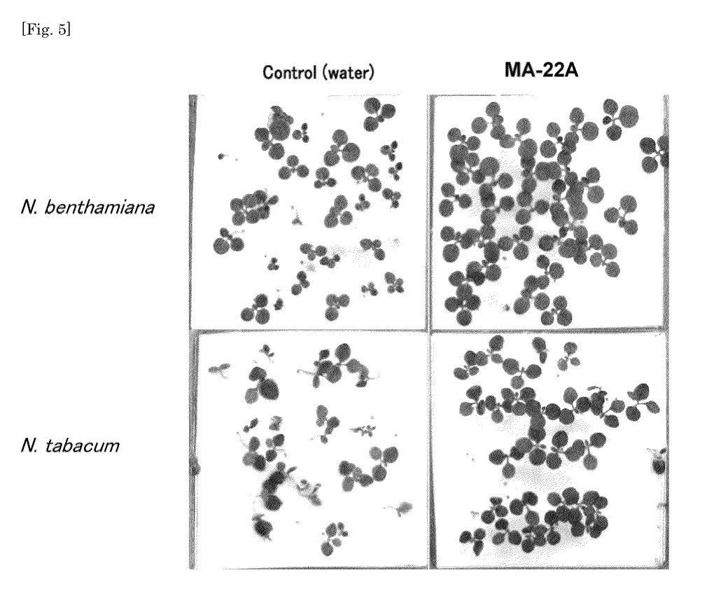

A typical example of the state of growth of tobacco (Nicotiana tabacum and Nicotiana benthamiana) observed from above of the plant boxes is shown in FIG. 5. It is seen in the figure that growth is promoted in the tobacco which was inoculated with bacteria MA-22A as compared to control.

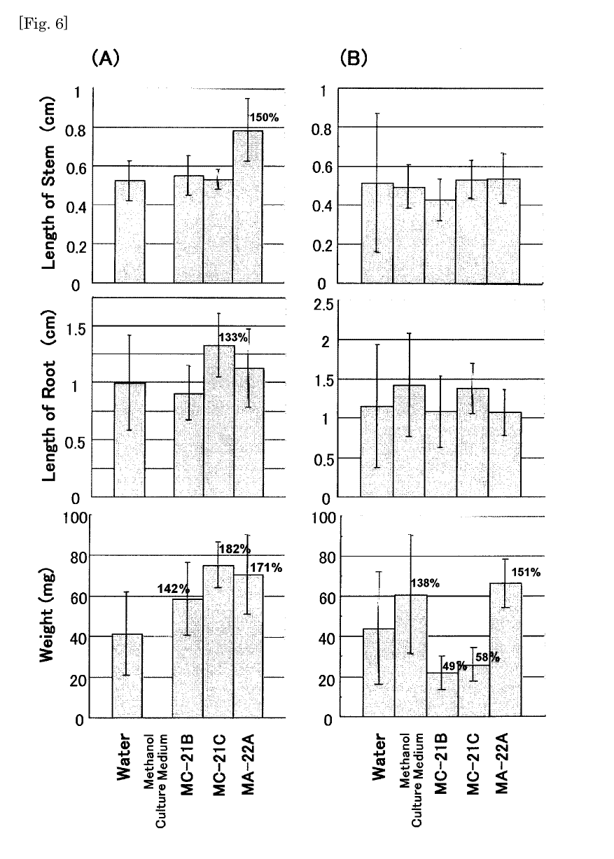

Also, the results of measurement of stem length, root length, and seedling weight of the N. tabacum plant bodies are shown in FIG. 6(A) with graphs. In the Figure, “Length of Stem” shows the length of plant body from seed surface to cotyledon. Among the seeds of N. tabacum, the group of seeds on which methanol culture medium (without bacterium strain) was applied did not germinate, but the group of seeds on which culture medium of MA-22A was applied showed a remarkable growth promotion accompanied with very great extension of stems as compared to control (water). Such effect was not seen in MC-21 or MC-22B. Also, as to whole body weight, it was remarkably increased in the group on which culture of MA-22A was applied, and the degree of the increase was equal to that in the group on which MC-21 or MC-22B was applied.

Also, results with the seeds of N. benthamiana are shown in FIG. 6(B). Among N. benthamiana, it is observed that the weight of the seedlings in the group in which MC-21 or MC-22B was applied on the seeds was only about half of control (water), whereas weight in the group of seeds on which culture medium of MA-22A was applied was remarkably increased.

### Example 3

### Growth Promotion of Barley

Seeds of barley (Hordeum vulgare, subsp. vulgare cultivar Akashinriki: provided from The Barley and Wild Plant Resources Research Center, The Research Institute for Bioresources, Okayama University) were treated with 70% ethanol for one minute, and then with 2% hypochlorous acid solution (containing approximately 1% Tween20) for 30 minutes, and were washed 4-5 times with sterilized water. 20 seeds each of the above seeds were placed on a solidified agar which had been prepared by pouring 40 ml of sterilized 0.8% agar into a separately sterilized plant box (40 seeds for each group). The culture of MC-21C or MA-22A which had been cultured according to the above method was dropped by 5 μL each on each of the seeds of a corresponding group, while only water or methanol culture medium was dropped on the seeds of control groups. The seeds were cultured at 25° C. for 8 days under light of a fluorescent lamp for 12 hours per day and in the dark for the rest of 12 hours, and then length of roots, length of parts above ground (leaf) and weight of seedlings were measured.

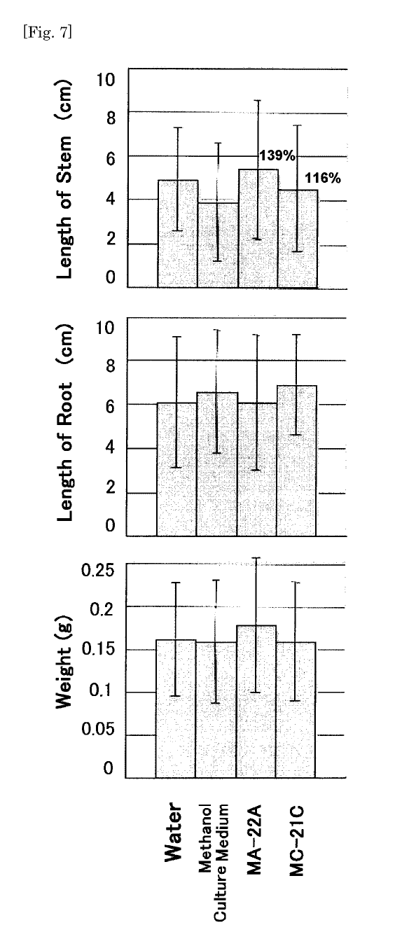

The results are shown in FIG. 7. In the seeds with which culture medium of MA-22A was in contact, it is observed that length of stems and weight were increased as compared to the control group and the group on which MC-21C was applied.

### Example 4

### Growth Promotion of Soybean

Fifty ml each of 0.8% agar, ½ MS culture medium were added to a sterilized plant box and allowed to solidify. Seeds of soybean (Glycine max, cultivar Enrei) were sterilized according to the following method, and planted in the above solidified culture medium (seven seeds/box). 2 pots were used for each sample. Bacteria suspension of MA-22A or MC-21C (the suspension which was prepared by culturing them in 5 mL of the methanol liquid culture medium with shaking at 28° C. for 5 days, and after centrifugation at 13000 rpm for five minutes and washing with sterilized water twice, suspending the bacteria in 5 mL final volume of sterilized water) were applied in each box by 20 μL per seed of soybean. Two boxes in which only water was applied instead of bacteria suspension were prepared as control. The seeds were left to stand at 4° C. for two days to allow them to absorb water. Then, the seeds were cultured at 25° C. in an incubator for plant cultivation, and 13 days later, length of roots and leaves and weight of the plant bodies (seedlings) were measured.

### <Method for Sterilization of Soybean Seeds>

The seeds were put in a tube of 50 mL, and washed well with water, and treated with 70% ethanol for three minutes. Then, ethanol was removed therefrom, and the seeds were soaked in 0.1% Tween20 aqueous solution containing 3% sodium hypochlorite for 30 minutes, and then washed with sterilized water four times in a clean bench.

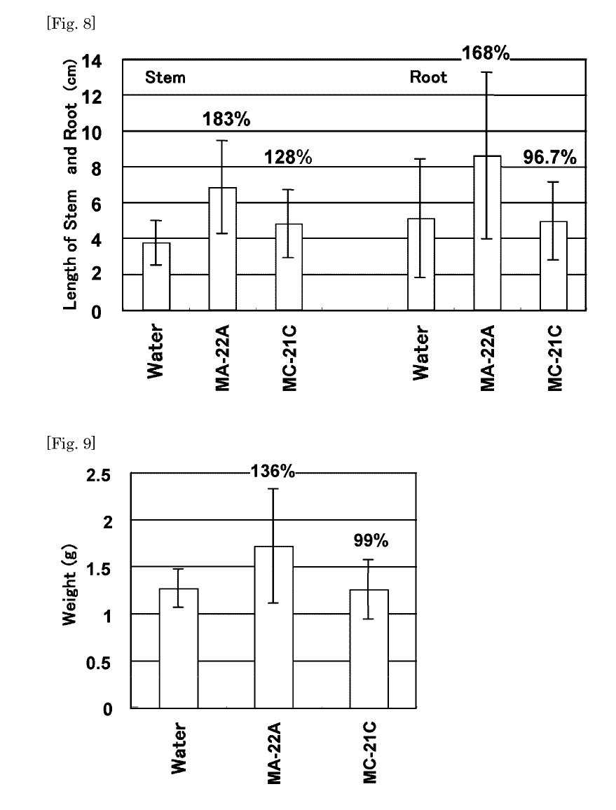

Results of the culture are shown in FIGS. 8 and 9. As shown in these figures, it is observed that length of stems, length of leaves, and weight of seedlings have remarkably increased in the soybeans which were inoculated with MA-22A.

### Example 5

### Growth-Promotion of Racomitrium canescens (2)

Ten g/L of sucrose was added to the following Knop+B5 vitamin culture medium, and after the pH of the mixture was adjusted to 5.8, 1.0 w/v % agar was added to prepare a culture medium for Racomitrium canescens.

TABLE 15
Knop + B5 Vitamin Culture Medium (in 100 mL)

| Component | Quantity Contained (mg) |
|---|---|
| KH₂PO₄ | 25 |
| MgSO₄•7H₂O | 25 |
| KCl | 25 |
| Ca(NO₃)•4H₂O | 100 |
| FeSO₄•7H₂O | 1.25 |
| Thiamine Hydrochloride | 1.0 |
| Myo-Inositol | 10 |
| Pyridoxine Hydrochloride | 0.1 |
| Nicotinic Acid | 0.1 |

Protonemata of Racomitrium canescens were cultured for about 1.5 months at 23° C. under light for 24 hours a day in a 300-mL conical flask containing 100 mL of the culture medium for Racomitrium canescens described above, and this brought a state where the surface of the culture medium was covered with Racomitrium canescens. The Racomitrium canescens was collected from the surface of the culture medium, rinsed with tap water, and two flasks of it were put in a blender (mfd. by Waring) containing 45 mL of purified water, and was broken down into fragments at 10000 rpm for 60 seconds. One mL of this liquid containing fragmented Racomitrium canescens was applied on culture soil prepared by molding vermiculite with cellulose fibers (Florialite: San-ei Co., Ltd). The Florialite had been sterilized by autoclaving before use. After the liquid containing fragmented Racomitrium canescens was applied, the Florialite was saturated with a diluted commercially available liquid fertilizer (Brand name Hyponex). Methylotrophic bacteria, MA-22A, MC-11A and MC-21C, which had been cultured to cover R2A agar medium in the petri dishes were used as inoculation sources. The bacteria were collected with a spatula, suspended in 500 μL of sterilized water, and then 50 μL of the suspension were inoculated to each Florialite to which the liquid of fragmented Racomitrium canescens had been applied. Moss was prepared which had been inoculated with no bacteria and was called Control 1, and, also, moss was prepared to which liquid fertilizer containing 3 mg/L kinetin had been applied without inoculation with bacteria was called Control 2. After the transplanting, all the Racomitrium canescens was acclimated to the same condition in an acclimating room with lighting apparatus while being humidified in order not to be dried.

After two weeks from the start of acclimation, the moss which had been inoculated with methylotrophic bacterium still kept their green color that was brighter than either of Control 1 or 2. In Control 1, the survival rate of the moss was low, and the gametophore formation was very limited. On the other hand, in Control 2, which received kinetin treatment alone, the survival rate of the moss was higher than Control 1, and gametophore formation was also well induced. Also, in the moss which was inoculated with the methylotrophic bacterium, survival rate was higher than Control 1, and gametophore formation was well induced like Control 2. Particularly, the moss inoculated with MC-11A strain showed the best growth and gametophore differentiation. Thus, survival rate and gametophore formation rate after acclimation were raised by inoculating methylotrophic bacterium.

### INDUSTRIAL APPLICABILITY

Since this invention enables fast growth of protonemata of Racomitrium canescens, it will open a way to an efficient and increased production of Racomitrium canescens, enabling a wide variety of utilizations of Racomitrium canescens as greening material. Also, since the invention promotes growth of tobacco, barley and soybean, it contributes to shortening the time period until their harvest as well as to increasing their yield.

1. A bacterium selected from methanol-utilizing bacteria of the genus Methylobacterium which bacteria are deposited under designated accession numbers FERM BP-11078, FERM BP-11079, FERM BP-11080 and FERM BP-11071, respectively.

2. A composition comprising bacterium of claim 1 and protonemata of Racomitrium canescens.

3. A composition according to claim 2, wherein the bacterium and the protonemata are contained in a culture medium.

4. A method for growth promotion of protonemata of Racomitrium canescens, which method comprises a step of providing at least one of methanol-utilizing bacteria of the genus Methylobacterium which bacteria are deposited under designated accession numbers FERM BP-11078, FERM BP-11079, FERM BP-11080, and FERM BP-11071, respectively, and a step of culturing protonemata of Racomitrium canescens together with the provided bacterium or bacteria.

5. A method for growth promotion of a seed plant selected from tobacco, barley and soybean, which method comprises a step of providing Methylobacterium aquaticum of methanol-utilizing bacterium deposited under designated accession No. FERM BP-11078, and a step of contacting the seeds of the selected seed plant with the bacterium, and then culturing the seeds.

6. A method according to claim 5, wherein the contact of the seeds with the bacterium is conducted by exposing the seeds to the culture medium of the bacterium.


SEQUENCE LISTING (9 sequences)

SEQ ID NO: 1 - DNA, 60 bp, Artificial
  Forward primer
cgcccgccgcgcgcggcgggcggggcgggggcacggggggactcctacgggaggcagcag

SEQ ID NO: 2 - DNA, 16 bp, Artificial
  Reverse primer
attaccgcggctgctg

SEQ ID NO: 3 - DNA, 1433 bp, Methylobacterium extorquens
ggctagagcgacgctggctgtaggcttatcacatgcttgtcgaacgggcaccttcgggtgtcagtggcagacgggtgagtaacacgtgggaacgtacccttcggttcggaataactcagggaaacttgagctaataccggatacgcccttttggggaaaggtttactgccgaaggatcggcccgcgtctgattagcttgttggtggggtaacggcctaccaaggcgacgatcagtagctggtctgagaggatgatcagccacactgggactgagacacggcccagactcctacgggaggcagcagtggggaatattggacaatgggcgcaagcctgatccagccatgccgcgtgagtgatgaaggccttagggttgtaaagctcttttgtccgggacgataatgacggtaccggaagaataagccccggctaacttcgtgccagcagccgcggtaatacgaagggggctagcgttgctcggaatcactgggcgtaaaagggcgcgtaggcggccgattaagtcgggggtgaaagcctgtggctcaaccacagaattgccttcgatactggttggcttgagatcggaagaggacagcggaactgcgagtgtagaggtgaaattcgtagatattcgcaagaacaccagtggcgaaggcggctgtctggtccggttctgacgctgaggcgcgaaagcgtggggagcaaacaggattagataccctggtagtccacgccgtaaacgatgaatgccagccgttggcctgcttgcaggtcagtggcgccgctaacgcattaagcattccgcctggggagtacggtcgcaagattaaaactcaaaggaattgacgggggcccgcacaagcggtggagcatgtggtttaattcgaagcaacgcgcagaaccttaccatcccttgacatggcatgttacctcgagagatcggggatcctcttcggaggcgtgcacacaggtgctgcatgggctgtcgtcagctcgtgtcgtgagatgttgggttaagtccccaacgagcgcaatcccacgtccttagttgccatcattcagttgggcactctatggagactgccggtgataagccgcgaggaaggtgtggatgacgtcaagtcctcatggcccttacgggatgggctacacacgtgctacaatggcggtgacagtgggacgcgaagccgcgaggtggagcaaatccccaaaaaccgtctcagttcggattgcactctgcaactcgggtgcatgaaggcggaatcgctagtaatcgtggatcagcacgccacggtgaatacgttcccgggccttgtacacaccgcccgtcacaccatgggagttggtcttacccgacggcgctgcgccaaccgcaaggaggcaggcgaccacggtagggtcagcgactggggtgaagtcgtaacaaggtagcc

SEQ ID NO: 4 - DNA, 1446 bp, Methylobacterium extorquens
agagtttgatcctggctcagagcgaacgctggcggcaggcttaacacatgcaagtcgaacgggcaccttcgggtgtcagtggcagacgggtgagtaacacgtgggaacgtacccttcggttcggaataactcagggaaacttgagctaataccggatacgcccttttggggaaaggtttactgccgaaggatcggcccgcgtctgattagcttgttggtggggtaacggcctaccaaggcgacgatcagtagctggtctgagaggatgatcagccacactgggactgagacacggcccagactcctacgggaggcagcagtggggaatattggacaatgggcgcaagcctgatccagccatgccgcgtgagtgatgaaggccttagggttgtaaagctcttttgtccgggacgataatgacggtaccggaagaataagccccggctaacttcgtgccagcagccgcggtaatacgaagggggctagcgttgctcggaatcactgggcgtaaagggcgcgtaggcggccgattaagtcgggggtgaaagcctgtggctcaaccacagaattgccttcgatactggttggcttgagaccggaagaggacagcggaactgcgagtgtagaggtgaaattcgtagatattcgcaagaacaccagtggcgaaggcggctgtctggtccggttctgacgctgaggcgcgaaagcgtggggagcaaacaggattagataccctggtagcccacgccgtaaacgatgaatgccagccgttggcctgcttgcaggtcagtggcgccgctaacgcattaagcattccgcctggggagtacggtcgcaagattaaaactcaaaggaattgacgggggcccgcacaagcggtggagcatgtggtttaattcgaagcaacgcgcagaaccttaccatcccttgacatggcatgttacctcgagagatcggggatcctcttcggaggcgtgcacacaggtgctgcatggctgtcgtcagctcgtgtcgtgagatgttgggttaagtcccgcaacgagcgcaacccacgtccttagttgccatcattcagttgggcactctagggagactgccggtgataagccgcgaggaaggtgtggatgacgtcaagtcctcatggcccttacgggatgggctacacacgtgctacaatggcggtgacagtgggacgcgaagccgcgaggtggagcaaatccccaaaaaccgtctcagttcggattgcactctgcaactcgggtgcatgaaggcggaatcgctagtaatcgtggatcagcacgccacggtgaatacgttcccgggccttgtacacaccgcccgtcacaccatgggagttggtcttacccgacggcgctgcgccaaccgcaaggaggcaggcgaccacggtagggtcagcgactggggtgaagtcgtaacaaggtagcc

SEQ ID NO: 5 - DNA, 544 bp, Spirosoma sp.
agagtttgatcctggctcaggatgaacgctagcggcaggcctaatacatgcaattcgaacgggtcgcaagaccagtggcaaacgggtcgcgtaacgcgtaagcaacctgcctcatactgggggatagcccggcgaaagctggggtaaacccgcacggtccgattgagtcacctggctcgatgggtaaacatttatgggtatgagaggggcttgcgtctgattagttagttggcagggtaacggcctaccaagacgatgatcagtaggggttctgaggggattggcccccacatgggtactgagatacggacccaactcctacgggaggcagcagtagggaatattgggcaatggaggcaactctgacccagccatgccgcgtgcaggatgaaggcgctcagcgttgtaaactgcttttattggtgaagaactgtagtcctgcgggattacttgacggtagccaaggaataagcaccggctaactccgtgccagcagccgcggtaatacggagggtgcaagcgttgtccggatttattgggttta

SEQ ID NO: 6 - DNA, 1424 bp, Methylobacterium extorquens
cgcctgtcggcaggctctaacacatgcaagtcgaactgggtaaccttcgggtgtcagtggcagactggtgagtaacacgtgggaacgtacccttcggttcggaataactcagggaaacttgagctaataccggatacgcccttttggggaaaggtttactgccgaaggatcggcccgcgtctgattagcttgttggtggggtaacggcctaccaaggcgacgatcagtagctggtctgagaggatgatcagccacactgggactgagacacggcccagactcctacgggaggcagcagtggggaatattggacaatgggcgcaagcctgatccagccatgccgcgtgagtgatgaaggccttagggttgtaaagctcttttgtccgggacgataatgacggtaccggaagaataagccccggctaacttcgtgccagcagccgcggtaatacgaagggggctagcgttgctcggaatcactgggcgtaaagggcgcgtaggcggccgattaagtcgggggtgaaagcctgtggctcaaccacagaattgccttcgatactggttggcttgagatcggaagaggacagcggaactgcgagtgtagaggtgaaattcgtagatattcgcaagaacaccagtggcgaaggcggctgtctggtccggttctgacgctgaggcgcgaaagcgtggggagcaaacaggattagataccctggtagtccacgccgtaaacgatgaatgccagccgttggcctgcttgcaggtcagtggcgccgctaacgcattaagcattccgcctggggagtacggtcgcaagattaaaactcaaaggaattgacgggggcccgcacaagcggtggagcatgtggtttaattcgaagcaacgcgcagaaccttaccatcccttgacatggcatgttacctcgagagatcggggatcctcttcggaggcgtgcacacaggtgctgcatggctgtcgtcagctcgtgtcgtgagatgttgggttaagtcccgcaacgagcgcaacccacgtccttagttgccatcattcagttgggcactctagggagactgccggtgataagccgcgaggaaggtgtggatgacgtcaagtcctcatggcccttacgggatgggctacacacgtgctacaatggcggtgacagtgggacgcgaagccgcgaggtggagcaaatccccaaaaaccgtctcagttcggattgcactctgcaactcgggtgcatgaaggcggaatcgctagtaatcgtggatcagcacgccacggtgaatacgttcccgggccttgtacacaccgcccgtcacaccatgggagttggccttacccgacggcgctgcgccaaccgcaaggaggcaggcgaccacggtagggtcagcgactggggtgaagtcgtaacaaggtaacc

SEQ ID NO: 7 - DNA, 1446 bp, Methylobacterium extorquens
agagtttgatcctggctcagagcgaacgctggcggcaggcttaacacatgcaagtcgaacgggcaccttcgggtgtcagtggcagacgggtgagtaacacgtgggaacgtacccttcggttcggaataactcagggaaacttgagctaataccggatacgcccttttggggaaaggtttactgccgaaggatcggcccgcgtctgattagcttgttggtggggtaacggcctaccaaggcgacgatcagtagctggtctgagaggatgatcagccacactgggactgagacacggcccagactcctacgggaggcagcagtggggaatattggacaatgggcgcaagcctgatccagccatgccgcgtgagtgatgaaggccttagggttgtaaagctcttttgtccgggacgataatgacggtaccggaagaataagccccggctaacttcgtgccagcagccgcggtaatacgaagggggctagcgttgctcggaatcactgggcgtaaagggcgcgtaggcggccgattaagtcgggggtgaaagcctgtggctcaaccacagaattgccttcgatactggttggcttgagaccggaagaggacagcggaactgcgagtgtagaggtgaaattcgtagatattcgcaagaacaccagtggcgaaggcggctgtctggtccggttctgacgctgaggcgcgaaagcgtggggagcaaacaggattagataccctggtagtccacgccgtaaacgatgaatgccagccgttggcctgcttgcaggtcagtggcgccgctaacgcattaagcattccgcctggggagtacggtcgcaagattaaaactcaaaggaattgacgggggcccgcacaagcggtggagcatgtggtttaattcgaagcaacgcgcagaaccttaccatcccttgacatggcatgttacctcgagagatcggggatcctcttcggaggcgtgcacacaggtgctgcatggctgtcgtcagctcgtgtcgtgagatgttgggttaagtcccgcaacgagcgcaacccacgtccttagttgccatcattcagttgggcactctagggagactgccggtgataagccgcgaggaaggtgtggatgacgtcaagtcctcatggcccttacgggatgggctacacacgtgctacaatggcggtgacagtgggacgcgaagccgcgaggtggagcaaatccccaaaaaccgtctcagttcggattgcactctgcaactcgggcgcatgaaggcggaatcgctagtaatcgtggatcagcacgccacggtgaatacgttcccgggccttgtacacaccgcccgtcacaccatgggagttggtcttacccgacggcgctgcgccaaccgcaaggaggcaggcgaccacggtagggtcagcgactggggtgaagtcgtaacaaggtagcc

SEQ ID NO: 8 - DNA, 1445 bp, Methylobacterium aquaticum
agagtttgatcctggctcagagcgaacgctggcggcaggcttaacacatgcaagtcgagcgggcccttcggggtcagcggcagacgggtgagtaacgcgtgggaacgtgcccttcggttcggaataactcagggaaacttgagctaataccggatacgcccttatggggaaaggtttatctgccgaaggatcggcccgcgtctgattagctagttggtgaggttacggctcaccaaggcgacgatcagtagctggtctgagaggatgatcagccacactgggactgagacacggcccagactcctacgggaggcagcagtggggaatattggacaatgggggcaaccctgatccagccatgccgcgtgagtgatgacggccttagggttgtaaagctcttttctccgggacgataatgacggtaccggaggaataagccccggctaacttcgtgccagcagccgcggtaatacgaagggggctagcgttgctcggaatcactgggcgtaaagggcgcgtaggcggctgatttaatcgagggtgaaagcccgtggctcaaccacggaatggccttcgatactgattggcttgagaccggaagaggacagcggaactgcgagtgtagaggtgaaattcgtagatattcgcaagaacaccagtggcgaaggcggctgtctggtccggttctgacgctgaggcgcgaaagcgtggggagcaaacaggattagataccctggtagtccacgctgtaaacgatgaatgctagccgttggggtgcatgcacctcagtggcgccgctaacgctttaagcattccgcctggggagtacggtcgcaagattaaaactcaaaggaattgacgggggcccgcacaagcggtggagcatgtggtttaattcgaagcaacgcgcagaaccttaccatcccttgacatggcatgcgagccggagagatccggtgttcccttcggggacgtgcacacaggtgctgcatggctgtcgtcagctcgtgtcgtgagatgttgggttaagtcccgcaacgagcgcaacccacgtcctcagttgccatcatttggttgggcactctggggagactgccggtgataagccgcgaggaaggtgtggatgacgtcaagtcctcatggcccttacggggtgggctacacacgtgctacaatggcggtgacaatgggcagcgaaggggcgacctggagcgaatccccaaaagccgtctcagttcggattgcactctgcaactcgggtgcatgaaggcggaatcgctagtaatcgtggatcagcatgccacggtgaatacgttcccgggccttgtacacaccgcccgtcacaccatgggagttggtcttacccgacggcgctgcgccaaccgcaaggaggcaggcgaccacggtagggtcagcgactggggtgaagtcgtaacaaggtagcc

SEQ ID NO: 9 - DNA, 621 bp, Mesorhizobium sp.
agagtttgatcctggctcagaacgaacgctggcggcaggcttaacacatgcaagtcgagcgcctcgcaagaggagcggcagacgggtgagtaacgcgtgggaatctacccatctctacggaacaactccgggaaactggagctaataccgtatacgtccttcgggagaaagatttatcggagatggatgagcccgcgttggattagctagttggtggggtaatggcctaccaaggcgacgatccatagctggtctgagaggatgatcagccacattgggactgagacacggcccaaactcctacgggaggcagcagtggggaatattggacaatgggcgaaagcctgatccagccatgccgcgtgagtgatgaaggccctagggttgtaaagctctttcaacggtgaagataatgacggtaaccgtagaagaagccccggctaacttcgtgccagcagccgcggtaatacgaagggggctagcgttgttcggaattactgggcgtaaagcgcacgtaggcggattgttaagttaggggtgaaatcccagggctcaaccctggaactgcctttaatactggcaatctcgagtccgagagaggtgagtggaattccgagtgta